(19) United States
(12) Patent Application Publication
(10) Pub. No.: US 2021/0100834 A1
(43) Pub. Date: Apr. 8, 2021

(54) COMPOSITION CONTAINING A CHLORIDE SALT FOR USE IN PREGNANT ANIMALS

(71) Applicant: Nutreco IP Assets B.V., Boxmeer (NL)

(72) Inventor: Pieter Langendijk, Amersfoort (NL)

(21) Appl. No.: 16/608,013

(22) PCT Filed: Apr. 24, 2018
(86) PCT No.: PCT/NL2018/050261

§ 371 Date: Oct. 24, 2019

(30) Foreign Application Priority Data
Apr. 25, 2017 (NL) NL2018777

(51) Int. Cl. A61K 33/06; A61K 33/42; A23L 33/00; A23K 20/24; A23K 20/26; A23L 33/16

(52) U.S. Cl. CPC A61K 33/06; A61K 33/42; A23L 33/16; A23K 20/24; A23K 20/26; A23L 33/30

(57) ABSTRACT

The disclosure pertains to a blood pH modulator for use in improving colostrum intake in offspring of an animal, in improving colostrum production by an animal, and in improving postnatal survival of offspring of an animal. This disclosure also pertains to compositions comprising a blood pH modulator in combination with a calcium binder to supplement a diet of an animal, in particular, a pregnant animal.

### CROSS-REFERENCE TO RELATED APPLICATIONS

This application is a national phase entry under 35 U.S.C. § 371 of International Patent Application PCT/NL2018/050261, filed, designating the United States of America and published as International Patent Publication WO 2018/050261 A1 on Apr. 24, 2018, which claims the benefit under Article 8 of the Patent Cooperation Treaty to Dutch Patent Application Serial No. NL2018777, filed Apr. 25, 2017.

### TECHNICAL FIELD

The disclosure in general pertains to the field of maximizing the reproductive performance of animals, in particular, mammals, specifically a swine. This disclosure, in particular, pertains to maximizing the performance of animals and their offspring by adapting their nutrition.

### BACKGROUND

Maximizing the performance of pregnant animals and their offspring has for a long time been a major objective of nutritionists. Inter alia feed-processing technologies, amino acid supplementation and increased dietary energy density have been used to address the objective of maximizing this performance. For example, research in dairy cattle, laying hens and swine has indicated positive effects on metabolism, in particular, reduced incidence of milk fever, greater egg shell quality and reduction of stillbirth, when the dietary electrolyte balance (dEB) was modified.

One important aspect of maximizing reproductive performance is optimizing the health of the pregnant animal and its (unborn) offspring during parturition. In particular, it is believed that this may reduce perinatal mortality of the offspring, such as stillbirth (intrapartum mortality) and neonatal mortality. In particular, stillbirth in piglets is mainly caused by oxygen insufficiency, due to repeated episodes of reduced blood perfusion of the placenta, caused by uterine contractions compressing the placenta, and due to stretch and sometimes rupture of the umbilical cord as the foetus travels through the uterus and the birth canal. The oxygen insufficiency leads to increasing blood lactate levels, a decreasing blood pH, and a reduction in extra-cellular fluid base excess in the foetus, a condition referred to as acidosis. When this condition aggravates, this can result in piglet death. Short term oxygen insufficiency is reversible; however, prolonged oxygen insufficiency may lead to irreversible effects and finally, death, and hence it is understandable that stillbirth occurs mainly in piglets born at the end of the birth order (see FIG. 1). Piglets that are born alive but have suffered from oxygen insufficiency can be permanently affected, which is reflected in their behaviour, reduced vigour and reduced performance.

It is commonly believed that one of the main causes for prolonged parturition is a reduced concentration of freely available calcium (ionic Calcium, or iCa) in maternal circulation. Calcium is involved in regulating the force and coordination of uterine contractions, and insufficient iCa in the blood results in poor muscle function, and as a consequence, prolonged parturition. For this reason, calcium supplementation to the diet of pregnant animals is often proposed as a measure to reduce perinatal mortality.

However, the influence of nutrition on maximizing performance, in particular, of reproductive performance, has not been conclusively explored yet.

### BRIEF SUMMARY

Described are methods and means to improve colostrum uptake of the offspring of an animal, particularly of piglets. It is also an object to improve colostrum production of an animal, in particular, a sow.

It has been found that it is advantageous to use a blood pH modulator such as a chloride salt for oral administration to maintain in a pregnant animal its blood pH at a physiological level. It appears that by maintaining the pH in the blood of the pregnant animal at its physiological level, perinatal mortality can be reduced, and colostrum uptake of neonates and production of a (lactating) animal can be improved. The basis for this finding is the recognition by applicant that pregnant animals with prolonged parturition, when compared to animals with uncomplicated parturitions, have a supranormal blood pH (for sows typically about 7.50 vs 7.45, i.e., in venous blood), and a concomitant lower than normal blood pCO₂, which phenomena are typical for respiratory alkalosis. The latter condition is believed to be caused by an increased respiratory rate in sows approaching parturition. It is believed that this hyperventilation causes increased evacuation of CO₂ from the circulation, causing the Henderson-Hasselbalch equation (H₂O+CO₂ ↑ [symbol] H₂CO₃ [symbol] H⁺HCO₃⁻) to shift to the left, with a drop in [H⁺] and a higher pH as a result. Some sows are apparently able to cope with the changes in acid-base balance, and maintain their blood pH, whereas some sows develop an increased pH. This increase in pH has been found to be directly related to perinatal mortality, in particular, to stillbirth and neonatal mortality of the offspring. In particular, it has been found that by maintaining the blood pH at its physiological level, the number of stillborn offspring of the pregnant animal and/or the percentage of animals having one or more stillborn offspring can be reduced. Also, colostrum production by the lactating animal and colostrum uptake of the offspring can be improved by applying the blood pH modulator. This may have a positive influence on improving postnatal survival of the offspring, in particular, neonatal survival of the offspring.

It is noted that in the art it is described to reduce the pH to a level below a physiological level in order to improve reproductive performance. DeRouchey et al. in Swine Research 2005, pp 34-37, shows that lowering the pH in the gastro intestinal tract and urine to below a normal level may be helpful, albeit to a very minor extent, to reduce the number of stillborn animals. In J. Anim. Sci. (2003, 81: 3167-3074), DeRouchey et al. show that a reduction of the blood pH to below a physiological level had a positive effect on piglet survivability, although no correlation could be found with litter and sow weights and the number of stillborn piglets. Elrod et al. in The Texas Journal of Agriculture and Natural Resources (2015, 28: 12-17) show that calcium ion supplementation for five days pre-farrowing may lead to a decrease of the urinary pH level below the physiological level of about 7.3 to a level as low as 5.65. At the same time, the duration of parturition and the number of stillbirths was also reduced. Elrod drew the conclusion that the data support the positive effect of calcium ion supplementation on swine parturition. It thus appears that the art is aiming mainly to positively influence the availability of iCa in the animal by decreasing the pH to a value below a physiological level.

### Definitions

A blood pH modulator is a compound, for example, a pure chemical substance or a mixture of substances that after digestion modulates the pH of the blood, for example, by lowering or raising the pH. Common blood pH modulators are inorganic substances such as minerals that alter the dietary electrolyte balance, and organic substances such as organic acids. Typically, a blood pH modulator is administered as a feed or drinking water additive, but it is noted that a particular feed (as a whole) can also be regarded as a “blood pH modulator” in the sense of the disclosure, for example, by extracting certain components in order to modulate the blood pH.

A physiological level of the blood pH of a pregnant animal is the pH that is characteristic of the healthy and normal functioning animal that lives of a normal diet. Healthy means that there is no significant negative effect on the health of the animal itself. Typically, a pregnant animal until a period of at least 5-10 days before parturition has a blood pH at physiological level, in particular, until at least 48 hours before parturition. For a pregnant sow, the physiological blood pH of venous blood is typically 7.45 with a standard deviation of 0.03 (within a group of animals).

An animal's diet is the habitual nourishment of the animal, including feed (solid and liquid feed) and drinking water.

A calcium binder is a compound that prevents calcium ions from being dissolved in water in a concentration of more than 10 mM, in particular, more than 1 mM.

A salt is insoluble in water if an aqueous solution of the salt contains less than 1 mmol of the salt per liter of water at room temperature.

A dietary supplement is a product intended for ingestion that contains a dietary ingredient intended to add nutritional value to the diet. The product can, for example, be added to the solid feed of the animals (in which case the supplement is often referred to as a top-dress) or to the drinking water (in which case the supplement is often referred to as a drinking water additive).

Perinatal mortality is the total of stillbirths and neonatal deaths.

Stillbirth is the phenomenon where offspring is born dead, but has died only shortly before (in particular, up to 24-48 hours) or during parturition.

Neonatal death is the phenomenon where offspring dies shortly after birth, typically within 10 days after birth.

Postnatal mortality refers to deaths occurring after birth but before weaning, and includes neonatal deaths.

Colostrum is the milk secreted between parturition and 24 h thereafter.

Colostrum uptake is calculated based on the increase in body weight between birth and 24 h thereafter.

### BRIEF DESCRIPTION OF THE DRAWINGS

FIG. 1 shows the percentage of piglets born alive or stillborn depending on the birth order. It demonstrates that stillbirth occurs mainly in piglets born at the end of the birth order.

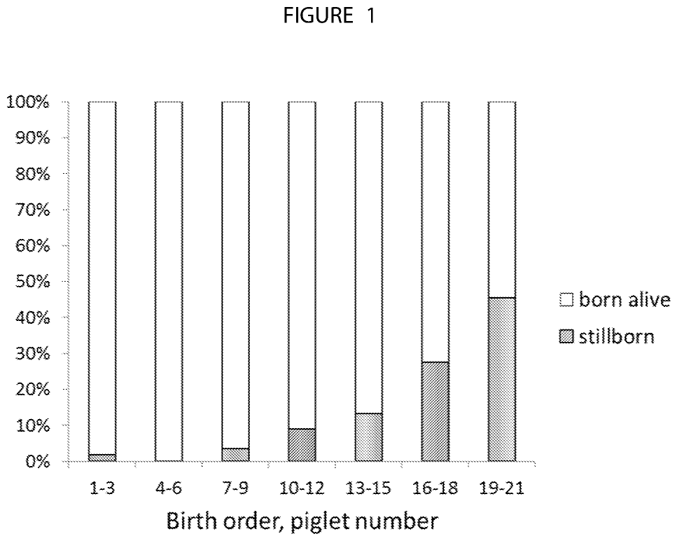

FIG. 2 shows the change in blood pH in pregnant sows approaching parturition.

FIG. 3 shows the blood pH for pregnant sows receiving a daily cation/anion balance of 270 mEq/d (“low dEB”) and 675 mEq/d (“high dEB”), respectively.

### DETAILED DESCRIPTION

The present disclosure provides a chloride salt for use in improving colostrum production of an animal, improving colostrum uptake in offspring of an animal, and improving neonatal/postnatal survival in the offspring of an animal.

The animal is preferably a pregnant animal, such as a non-human, pregnant animal, preferably a pregnant farming animal, even more preferably a pregnant monogastric farming animal, and most preferably a pregnant sow. As such, the offspring is preferably piglets.

The blood pH modulator may be used to maintain the pH at a physiological level within 0 to 36 hours before parturition and/or during parturition, in particular, within 0 to 24 hours before parturition and/or during parturition. It was found, in particular, for pregnant swine, that the pH levels typically increase to above a physiological level in the window of 0-36 hours, in particular, 0-24 hours before parturition. Ideally, the pH is maintained at a physiological level during this critical period.

In an embodiment, the blood pH modulator, such as a chloride salt, is added to the animal's diet (which also encompasses enriching the diet in certain components by extracting other ones). This was found to be a convenient way to provide for oral administration. The blood pH modulator, such as a chloride salt, may be added to the drinking water or feed of the animal. In particular, the blood pH modulator, such as chloride salt, may be added to the drinking water of the animal. The advantage thereof is that pregnant animals, even right before parturition, normally show regular or even increased drinking behaviour, whereas they might decrease or even cease the eating of solid feed. Thus, consumption of the blood pH modulator, such as chloride salt, may be continued particularly at the point in time when approaching parturition, which is when the respiratory rate in sows increases, and when the blood pH modulator is most effective.

In yet another embodiment, the blood pH modulator, such as chloride salt, is administered in a period of 0 to 5 days before parturition, in particular, at least in a period of 1 to 5 days before parturition (which does not exclude that the modulator is also administered earlier or later, for example, during parturition). It was found that by administering the blood pH modulator during this period, the pH may be kept at its physiological level before and potentially also during parturition.

In still another embodiment, the blood pH modulator comprises a mineral, in particular, a salt, specifically a chloride salt, such as ammonium chloride, or calcium chloride or organic chloride salts, such as betaine HCl, lysine HCl or choline chloride. In a particularly suitable and preferred embodiment, the blood pH modulator is a chloride salt as taught herein. It was shown that using the blood pH modulator, particularly a chloride salt, the pH of the blood can be modulated such that before and during parturition the pH remains at a physiological level.

In an embodiment, the blood pH modulator, e.g., chloride salt, is added to the animal's diet, e.g., by means of addition of the modulator to the drinking water or feed.

In an embodiment, the blood pH modulator, such as chloride salt, is administered to the animal in such an amount that the electrolyte balance dEB of the diet is in the range of about 0-400 mEq/day, such as in the range of about 25-375 mEq/day, about 50-350 mEq/day, about 75-325 mEq/day, about 100-300 mEq/day, about 125-275 mEq/day, or about 150-250 mEq/day.

The amount of the modulator, e.g., chloride salt that is added to the animal's diet in the form of feed may be such that it allows to obtain an electrolyte balance of 50 to 150 mEq/kg in the total diet. This means that in the total dietary intake the average value of the electrolyte balance may be between 50 and 150 mEq/kg. This may be any value such as 50, 55, 60, 65, 70, 75, 80, 85, 90, 95, 100, 105, 110, 115, 120, 125, 130, 135, 140, 145 and 150 mEq/kg of the total diet. As an example, when administering the blood pH modulating mineral in a top-dress that is dosed at 400 g per day, the value for the top-dress as such may be around −600 mEq/kg. This way, by administering the top-dress, the total daily diet will have a lowered electrolyte balance of about −240 mEq per day (which is about −100 mEq/kg assuming a total intake of 2.4 kg per day). For a calculation of the amount of mineral in mEq, referral is made to the paper by Elliot Block, Arm & Hammer Animal Nutrition, Princeton, N.J., USA, called “Revisiting Negative Dietary Cation-Anion Difference Balancing for Prepartum Cows and its Impact on Hypocalcaemia and Performance” (http://dairy.ifas.ufl.edu/rns/2011/5block.pdf), in particular, to page 35 where an example is given for a calculation of a mineral concentrations in mEq.

In a suitable embodiment, the blood pH modulator, in particular, chloride salt, is added to the drinking water of the animal, and the drinking water of the animal comprises between about −5 and −45 mEq/L, such as between about −10 and −40, between about −15 and −35, between about −15 and −30, or between about −15 and −25 mEq/L of the blood pH modulator, in particular, the chloride salt.

In an embodiment, the blood pH modulator, in particular, the chloride salt, is administered in a period of 0 to 5 days before parturition, in particular, at least in a period of 1 to 5 days before parturition.

In an embodiment, a calcium binder may additionally be added to the animal's diet, such as to the feed or to the drinking water. For example, the calcium binder may be contained in a composition, which is in the form of a premix, a liquid, a powder, granules, a pellet, a dragee, a tablet, a pill, or a capsule.

In a particularly suitable embodiment, the calcium binder may be added to the drinking water of the animal, for the same reason as mentioned above in respect of the blood pH modulator.

Completely contrary to the prior art teaching that it is advantageous to supplement the diet with extra calcium, it was found that the addition of a calcium binder to the diet has a positive effect on perinatal mortality, in particular, on a reduction of stillbirth. Without being bound to theory, it is believed that by effectively lowering the amount of available calcium in the animal's diet, the animal's natural capability to release calcium from its mineral reserves (in particular, from the bone) might be stimulated. In a particular embodiment, the calcium binder is an anion that forms a water insoluble salt with calcium ions in aqueous solution. Such an ion may, for example, be a conjugated base ion.

Suitable calcium binders include, without limitation, any compounds that are capable of binding free calcium in the ruminant gastro-intestinal tract whereby the free calcium cannot be absorbed by the animal. Thus, the natural calcium regulating defence mechanism of the animal is triggered. Such compounds include, without limitation, phosphoric acid, oxalic acid, sodium oxalate, phytic acid, a phytate, a clay mineral including zeolite, sodium diethylene acetic acid, ethylene diaminetetraacetic acid (EDTA), the sodium salts of EDTA Na2EDTA and Na4EDTA, trisodium nitrilotriacetate monohydrate, trisodium ntrioacetate, pentasodium diethylenetriaminepentaacetate, tri sodium N-hydroxyethylethylenediaminetriacetate, citric acid, a citrate, a polyphosphate, a tripolyphosphate, a phosphate, a cellulose phosphate, glutamic acid N,N-diacetic acid (GLDA), a sodium salt of GLDA, a potassium salt of GLDA, methylglycine-N,N-diacetic acid (MGDA), a sodium salt of MGDA, a potassium salt of MGDA, ethylenediamine N,N′-disuccinic acid (EDDS), a sodium salt of EDDS, a potassium salt of EDDS, iminodisuccinic acid (IDS), a sodium salt of IDS, or a potassium salt of IDS, or a calcium-free derivative of any such compounds.

The calcium binder may advantageously be phosphoric acid or a phosphate, particularly when the calcium binder is added to drinking water. Phosphoric acid has the interesting property that it does not form a water insoluble salt with calcium ions at low pH (i.e., when in solution prior to administration to an animal, it remains in soluble form, even when formulated with a calcium salt such as calcium chloride), whilst it precipitates with calcium ions at physiological pH (i.e., in the gastrointestinal tract of the animal). The calcium binder is preferably an anion that forms a water insoluble salt with calcium ions at physiological small intestinal pH (e.g., a pH in the range of 6-7.5, preferably 6.5-7.5) but not at acidic pH (e.g., a pH below 6, preferably below 5.5).

The calcium binder may be encapsulated by any appropriate encapsulating material. A compound suitable for encapsulation of calcium binder is a compound selected from the groups consisting of a fat, a non-calcium derivative of a fat such as a soap and a stearate, a protein, a polysaccharide, a cellulose and a derivative of any such compound, a gum, a glycol and gelatine.

The latter teaching of advantageously using an additional calcium binder, provides that the disclosure is also embodied in the use of a blood pH modulator such as a chloride salt in combination with a calcium binder such as phosphoric acid to supplement a diet of a pregnant animal in a period of 0 to 5 days before parturition and in a dietary supplement comprising in combination a blood pH modulator such as a chloride salt, e.g., calcium chloride, and a calcium binder such as phosphoric acid.

The addition of a calcium binder is particularly advantageous when calcium chloride is used as the chloride salt.

Compositions and Uses Thereof

The present disclosure teaches an aqueous composition comprising a chloride salt as taught herein such as calcium chloride and a calcium binder as taught herein such as phosphoric acid, particularly for feeding to a pregnant animal, preferably a pregnant farming animal, e.g., in the form of a supplement, e.g., by means of addition to the drinking water. Therefore, preferably such aqueous composition comprises as chloride salt calcium chloride and as calcium binder phosphoric acid.

In an embodiment, such aqueous composition comprises between about −5 and 45 mEq/L, such as between about −10 and −40, between about −15 and −35, between about 15 and −30, or between about −15 and −25 mEq/L of the chloride salt, such as the calcium chloride.

In an embodiment, the aqueous composition further comprises between about 0.1 and about 5 g/L, such as between 0.25 and 4.5 g/L, between 0.5 and 4 g/L, between 0.5 and 3.5 g/L, between 0.6 and 3 g/L, between 0.7 and 2.5 g/L, or between 0.75 and 2 g/L of a calcium binder, in particular, of phosphoric acid.

Therefore, in a highly suitable embodiment the aqueous composition taught herein comprises between about −5 and −45 mEq/L, such as between about −10 and −40, between about −15 and −35, between about −15 and −30, or between about −15 and −25 mEq/L of calcium chloride, and between about 0.1 and about 5 g/L, such as between 0.25 and 4.5 g/L, between 0.5 and 4 g/L, between 0.5 and 3.5 g/L, between 0.6 and 3 g/L, between 0.7 and 2.5 g/L, or between 0.75 and 2 g/L of phosphoric acid.

In an embodiment, the aqueous composition taught herein is intended for administration as drinking water to an animal, particularly a pregnant animal, such as a pregnant sow.

In an embodiment, it is intended for administration to the animal in a period of 0 to 5 days before parturition, in particular, at least in a period of 1 to 5 days before parturition.

The present disclosure also provides a concentrated version of the aqueous composition taught herein, referred to as an aqueous supplement composition, which may commercially be sold for further dilution, such as intended for 1% dilution, into drinking water. In such case, the farmer will ensure dilution on-farm prior to administration thereof, resulting in the aqueous composition taught hereinabove.

The present disclosure therefore also provides an aqueous supplement composition that upon dilution in drinking water provides the aqueous composition taught hereinabove. In an embodiment, such aqueous supplement composition is suitable for 1% dilution into drinking water.

Thus, such aqueous supplement composition, when intended for 1% dilution into drinking water, may comprise between about −500 and −4500 mEq/L, such as between about −1000 and −4000, between about −1500 and −3500, between about −1500 and −3000, or between about 1500 and −2500 mEq/L of a chloride salt, preferably calcium chloride, and between about 10 and about 500 g/L, such as between 25 and 450 g/L, between 50 and 400 g/L, between 50 and 350 g/L, between 60 and 300 g/L, between 70 and 250 g/L, or between 75 and 200 g/L of a calcium binder, in particular, phosphoric acid.

In a highly suitable embodiment, the aqueous supplement composition, when intended for 1% dilution into drinking water, comprises between about 50 and about 250 g/L calcium chloride, and between about 50 and about 250 g/L phosphoric acid.

Of course, the abovementioned amounts of chloride salt and calcium binder, in particular, of calcium chloride and phosphoric acid, in such aqueous supplement composition may differ depending on the degree of dilution to be applied. The skilled person is capable of adjusting the amounts depending on the desired degree of dilution.

The compositions taught herein may further comprise at least one further ingredient such as a vitamin, a mineral, and a carrier. Further ingredients may be selected from a sugar, a stabilising agent, and a colouring agent.

This disclosure will now be further explained using the following non-limiting examples.

### EXAMPLES

### Example 1

In a first series of experiments, the relationship between blood pH and reproductive performance was examined. Based on these experiments it was shown that a small deviation (for example, 0.03 units) from a physiological pH level may have great impact on physiological processes as established by impact on mortality, in particular, stillbirth, but also on the health of piglets born alive, not only showing through a lower postnatal mortality but also by showing an increased colostrum intake. Subtle changes in pH value appear to be associated with an increase in percentage of stillborn animals and appear to affect vitality of the piglets, time they take to have their first suckle, and consequently mortality rate.

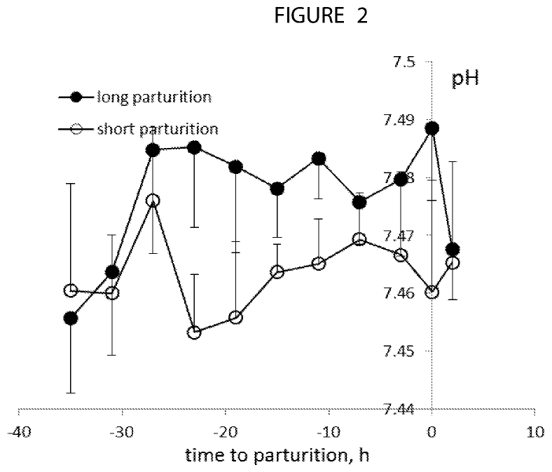

The effect of pH on stillbirth is illustrated by the fact that sows with prolonged parturition appear to have an increased pH (respiratory alkalosis) and an increased stillbirth rate. In a first experiment, 39 sows were divided in three classes of duration of parturition (14 animals had a duration of parturition >300 minutes; 10 animals had a duration of parturition of 200-300 minutes and 15 animals had a duration of parturition of <200 minutes). The number of stillborn animals varied from 1.6 (long duration), via 1.0 (intermediate duration) to 0.5 (short duration) while the pH varied from 7.49 (increased level) to 7.46 (physiological level) for these animals at start of parturition. The change in blood pH in sows approaching parturition is indicated in FIG. 2. Initially blood pH is similar, both at a physiological level of 7.46 (for these animals), however, sows that have prolonged parturition develop respiratory alkalosis (increased blood pH) in the period prior to parturition, whereas sows with normal birth show a normal (physiological) pH.

The relationship between maternal blood pH and condition of piglets is further illustrated in an experiment wherein for 94 sows, the blood pH and lactate level for piglets born to 47 sows with increased blood pH were compared to the blood pH and lactate levels of piglets born to 47 sows with pH at physiological level (see table 1).

TABLE 1
pCO2 (mm Hg), pH and lactate (mmol/l) in piglets to sows
with physiological and increased blood pH (>7.47)

| | born to sows with | born to sows with |
|---|---|---|
| Piglet blood values | normal blood pH | increased blood pH |
| pCO2 | 43.3 | 45.1 |
| pH | 7.41 | 7.34 |
| Lactate | 5.48 | 7.49 |

As can be seen in table 1, piglets born to sows with increased pH had a lower blood pH and higher lactate, indicating that these piglets had suffered more from oxygen insufficiency (hypoxia) during birth. This indicates that parturition for these piglets was compromised and as a consequence they had a higher stillbirth rate, and for those piglets surviving, a poorer condition at birth (clinical signs of hypoxia).

In yet another experiment it was assessed whether it would be possible to maintain the blood pH level at a physiological level by using as a pH modulator a mineral. It is known that in general blood pH can be modulated by changing the dietary electrolyte balance, or cation/anion balance of the diet. This is the difference between the total of cations and anions in the diet, and is calculated (in this experiment) as the balance between molar content of Na⁺+K⁺−Cl⁻(see Block reference as mentioned here above). In the experiment a lower than normal electrolyte balance through the diet was achieved by including relatively more Cl⁻ ions in the diet than Na⁺ and K⁺ ions. A normal diet typically contains 200 to 300 mEq/kg, so at 2.5 kg intake the daily cation/anion balance would be 500 mEq/d to 750 mEq/d.

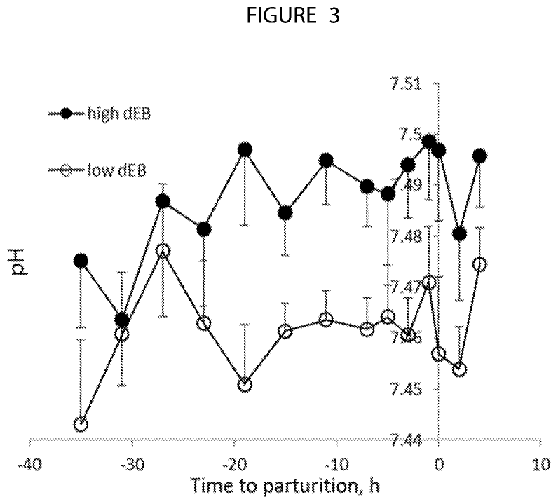

A diet was formulated to contain a cation/anion balance of 100 mEq/kg and compared to a standard diet with a cation/anion balance of 250 mEq/kg. The diets were fed from 5 days prior to farrowing at 2.7 kg per day, until the sows had farrowed, resulting in a daily cation/anion balance of 270 mEq/d and 675 mEq/d respectively. The blood pH for these sows is shown in FIG. 3. As can be seen, a normal (physiological) blood pH for sows on the 100 mEq/kg diet was obtained, whereas in the control group fed a regular diet the blood pH level was increased to above a physiological level prior to farrowing. In sows fed the 100 mEq/kg diet, stillbirth rate was reduced from 1.8 stillborn per litter to 0.8 stillborn piglets per litter. Litter size was 15.5 total born (alive and stillborn) piglets on average.

In another experiment, cations and anions were added to drinking water in such a balance, that the daily cation/anion balance of diet plus drinking water was reduced. For this, the drinking water contained 1.5 g calcium chloride per liter. The diets fed to the sows were the same, and hence the reduction in daily cation/anion balance was solely caused by the drinking water treatment. The cation/anion balance was reduced from 540 mEq/d in controls (normal water) to 100 to 400 mEq/d in sows with treated water; the variation in the latter was caused by variation in water intake. In this experiment, next to the calcium chloride to reduce the daily cation/anion balance, a calcium binder (phosphoric acid, PhA)) was added in a concentration of 0.01 mol/liter. The results of the study are indicated here below in table 2.

TABLE 2
Effects of pH modulator and optional
calcium binder on perinatal phenomena

| | CaCl₂ + PhA* | PhA* | Control |
|---|---|---|---|
| Number of sows | 28 | 25 | 24 |
| Stillborn piglets, # | 1.0 | 1.1 | 1.6 |
| Average colostrum intake | 447 | 408 | 422 |
| per piglet, g/24 h | | | |
| Piglets with colostrum | 7.1% | 15.1% | 13.9% |
| intake <250 g/24 h | | | |
| Postnatal mortality | 5.2% | 9.4% | 8.0% |

*PhA = phosphoric acid

In this experiment, stillbirth was reduced from 1.6 to 1.0 piglets per litter. It was also confirmed that next to reducing stillbirth, the condition of piglets born alive could be improved. This is reflected in the uptake of colostrum, which was increased from 422 to 447 g/piglet (calculated on the basis of weight gain) in the first 24 h of life, even though the total number of piglets born alive was also increased from 14.1 to 14.7. More importantly, the percentage of piglets with an intake lower than 250 g colostrum, a threshold deemed critical for neonatal survival, was reduced from 14% to 7%. In addition to reducing stillbirth, the disclosure also decreased neonatal mortality from 8% to 5%. Remarkably, the addition of a calcium binder had no negative effect on perinatal mortality. On the contrary, given the result for the calcium binder alone on stillbirth, it is expected that the calcium binder has even increased the positive effects of the calcium chloride. It is believed that binding of dietary calcium in the gut and the inherent effect of reducing the amount of calcium available to the animal has as a consequence that the animal is triggered to mobilise calcium from its own mineral reserves in the bones. Therefore, when demand for calcium is increased as in parturition, the animal is more prepared to face this demand, and better equipped to maintain blood Ca levels. In the examples described above, blood calcium levels were indeed increased by the treatments provided (data not shown).

### Example 2

In another example, a pH blood modulator (calcium chloride) was mixed into a top-dress consisting of rice bran, which contains a calcium binding acid, namely phytic acid. The top-dress was fed from 5 days before farrowing, when the sows entered the farrowing room, until the end of parturition. The top-dress resulted in an overall electrolyte balance of the diet of 140 mEq/kg compared to 200 mEq/kg in the control group. Both groups received a total of 2.7 kg feed per sow per day. Stillbirth was reduced by 0.6 piglet per litter in the top-dress treatment (see Table 3).

TABLE 3
Effect of a supplement containing a blood pH modulator
and a calcium binding acid (rice bran containing
phytic acid) on the outcome of parturition

| | dEb 200 mEq/kg | dEb 140 mEq/kg |
|---|---|---|
| N (number of sows) | 23 | 38 |
| Born alive | 14.1^{a} | 14.7^{b} |
| Stillborn | 1.5^{a} | 0.9^{b} |
| Sows with >1 stillborn | 71% | 44% |
| and/or assistance* | | |

*Manual assistance in delivery or palpation;
^{a,b}P < 0.05

### Example 3

In another trial the effect of modulating the pH in pregnant sows to physiological levels was examined, in particular, the effect on pH itself and the perinatal mortality. For the trial periparturient sows were allocated to one of two treatments at 1 week before parturition. The treatments were either a high dietary electrolyte balance (250 mEq/kg) or a low dietary electrolyte balance (100 mEq/kg) by adding calcium chloride. The two diets were fed (±2.6 kg per day) until parturition had finished. All sows received ear vein catheters at least three days before parturition to allow frequent blood sampling to measure the blood pH. Only animals with good general health based on adequate feed intake, good locomotory condition and good general visual presentation were included for assessing the results. These results are indicated in table 4.

TABLE 4
Effect on stillbirth of diets differing in electrolyte balance

| | 250 mEq/kg | 100 mEq/kg |
|---|---|---|
| | (high) | (low) |
| Number of sows | 21 | 21 |
| Blood pH of the sows, | 7.51 | 7.46 |
| 12-24 h pre-farrowing | | |
| Total born piglets | 15.7 | 15.4 |
| Born alive | 13.9^{a} | 14.6^{b} |
| Stillborn | 1.8^{a} | 0.8^{b} |
| Litters with 0 or 1 stillborn | 11 | 16 |

^{a,b}P < 0.05

As can be seen, with the low dEB diet (i.e., 100 mEq/kg total diet), blood pH of the sows could be maintained at the physiological level, whereas with the high dEB diet, the blood pH in a period 12 to 24 hours before parturition increased to 7.51 (P<0.01).

The effect on the total number of animals born and the number of animals born alive was not statistically significant. However, the number of stillborn piglets was decreased significantly (P=0.09) in the sows wherein the blood pH was maintained at a physiological level. Also, the number of litters with no or at most 1 stillborn piglet was increased significantly (P=0.07).

1.-19. (canceled)

20. A method of treating an animal so as to improve colostrum production of the animal, improve colostrum uptake of an offspring of the animal, improve postnatal survival of the animal's offspring, and/or increase the number of litters of the animal with no or at most one (1) stillborn offspring, the method comprising:
administering a chloride salt to the animal; and
so as to improve colostrum production of the animal, improve colostrum uptake of the animal's offspring, improve postnatal survival of the animal's offspring, and/or increase the number of litters of the animal with no or at most one (1) stillborn offspring.

21. The method according to claim 20, wherein the chloride salt is added to the diet of the animal.

22. The method according to claim 20, wherein the chloride salt is added to the drinking water or feed of the animal.

23. The method according to claim 21, wherein the chloride salt is administered to the animal in such an amount that the electrolyte balance dEB of the diet is about 0-400 mEq/day.

24. The method according to claim 21, wherein the chloride salt is added to the animal's diet in such an amount as to obtain an electrolyte balance of 50 to 150 mEq/kg in the total diet.

25. The method according to claim 22, wherein the chloride salt is included in the drinking water of the animal so that the drinking water has between about −15 and −45 mEq/liter of the chloride salt.

26. The method according to claim 20, wherein the chloride salt is administered in a period of 0 to 5 days before parturition of the animal.

27. The method according to claim 26, wherein the chloride salt is administered at least in a period of 1 to 5 days before parturition.

28. The method according to claim 20, wherein the chloride salt comprises an ammonium chloride, a calcium chloride, a betain hydrochloride, a lysine hydrochloride, or a choline chloride.

29. The method according to claim 20, wherein a calcium binder is additionally added to the animal's diet.

30. The method according to claim 29, wherein the calcium binder is an anion that forms a water insoluble salt with calcium ions.

31. An aqueous composition comprising:
between about −5 and −45 mEq/L of a chloride salt, and
between about 10 and about 500 g/L of a calcium binder.

32. The aqueous composition of claim 31, wherein the chloride salt comprises an ammonium chloride, a calcium chloride, a betain hydrochloride, a lysine hydrochloride, or a choline chloride.

33. The aqueous composition of claim 31, wherein the calcium binder is an anion that forms a water insoluble salt with calcium ions.

34. The aqueous composition of claim 31, wherein the aqueous composition is drinking water for administration to an animal in such an amount that the electrolyte balance dEB of the diet of the animal is about 0-400 mEq/day.

35. A method of treating an animal about to undergo parturition, the method comprising:
administering to the animal the aqueous composition of claim 31 in a period of 0 to 5 days before parturition.

36. The method according to claim 35, wherein the aqueous composition is administered at least in a period of 1 to 5 days before parturition.

37. The method according to claim 35, wherein chloride salt is administered to the animal in such an amount that the electrolyte balance dEB of the diet is about 0-400 mEq/day.

38. The method according to claim 35, wherein the animal is a pregnant sow.

39. An aqueous supplement composition suitable for 1% dilution into drinking water, the aqueous supplement composition comprising:
between about −500 and −4500 mEq/L of a chloride salt, and
between about 10 and about 500 g/L of a calcium binder.

40. The aqueous supplement composition of claim 39, which comprises:
between about 50 and about 250 g/L calcium chloride, and
between about 25 and about 150 g/L phosphoric acid.
